(19) United States
(12) Patent Application Publication
(10) Pub. No.: US 2005/0214913 A1
(43) Pub. Date: Sep. 29, 2005

(54) Method for producing L-amino acids by fermentation using bacteria having enhanced expression of xylose utilization genes

(76) Inventors: Aleksey Nikolaevich Marchenko, Moscow (RU); Sergey Vladimirovich Benevolensky, Moscow (RU); Elena Vitalievna Klyachko, Moscow (RU); Yuri Ivanovich Kozlov, Moscow (RU)

Correspondence Address: CERMAK & KENEALY LLP;ACS LLC, 515 EAST BRADDOCK ROAD, SUITE B, ALEXANDRIA, VA, 22314, US

(21) Appl. No.: 11/059,686

(22) Filed: Feb. 17, 2005

Related U.S. Application Data
(60) Provisional application No. 60/610,545, filed on Sep. 17, 2004.

(30) Foreign Application Priority Data
Mar. 16, 2004 (RU) 2004107548

(51) Int. Cl. C12P 13/24; C12N 1/21

(52) U.S. Cl. 435/107; 435/252.33

(57) ABSTRACT

A method for producing an L-amino acid, such as L-histidine, using bacterium belonging to the genus Escherichia having increased expression amount of genes, such as xylABFGHR locus, coding for xylose utilization enzymes, is disclosed. The method comprises cultivating the L-amino acid producing bacterium in a culture medium containing xylose, and collecting the L-amino acid from the culture medium.

This application claims the benefit of U.S. provisional patent application No. 60/610,545 filed on Sep. 17, 2004, under 35 USC §119(e).

### BACKGROUND OF THE INVENTION

1. Technical field

The present invention relates to a method for producing L-amino acids by pentose fermentation, and more specifically to a method for producing L-amino acids using bacteria having enhanced expression of xylose utilization genes by fermentation of mixture of arabinose and/or xylose along with glucose as a carbon source. The non-expensive carbon source which includes a mixture of hexoses and pentoses of hemicellulose fractions from cellulosic biomass can be utilized for commercial production of L-amino acids, for example, L-histidine.

2. Background art

Conventionally, L-amino acids have been industrially produced by fermentation processes using strains of different microorganisms. The fermentation media for the process typically contains sufficient amounts of different sources of carbon and nitrogen.

Traditionally, various carbohydrates such as hexoses, pentoses, trioses; various organic acids and alcohols are used as a carbon source. Hexoses include glucose, fructose, mannose, sorbose, galactose and the like. Pentoses include arabinose, xylose, ribose and the like. However, the above-mentioned carbohydrates and other traditional carbon sources, such as molasses, corn, sugarcane, starch, its hydrolysate, etc., currently used in industry are rather expensive. Therefore, finding alternative less expensive sources for production of L-amino acids is desirable.

Cellulosic biomass is a favorable feedstock for L-amino acid production because it is both readily available and less expensive than carbohydrates, corn, sugarcane or other sources of carbon. Typical amounts of cellulose, hemicellulose and lignin in biomass are approximately 40-60% of cellulose, 20-40% of hemicellulose 10-25% of lignin and 10% of other components. The cellulose fraction consists of polymers of a hexose sugar, typically glucose. The hemicellulose fraction is made up of mostly pentose sugars, including xylose and arabinose. The composition of various biomass feedstocks is shown in Table 1 (http://www.ott.doe.gov/biofuels/understanding_biomass.html)

TABLE 1

| | Six-carbon | | | |
|---|---|---|---|---|
| Material | sugars | Five-carbon sugars | Lignin | Ash |
| Hardwoods | 39-50% | 18-28% | 15-28% | 0.3-1.0% |
| Softwoods | 41-57% | 8-12% | 24-27% | 0.1-0.4% |

More detailed information about composition of over 150 biomass samples is summarized in the “Biomass Feedstock Composition and Property Database” (http://www.ott.doe.gov/biofuels/progs/search1.cgi).

An industrial process for effective conversion of cellulosic biomass into usable fermentation feedstock, typically a mixture of carbohydrates, is expected to be developed in the near future. Therefore, utilization of renewable energy sources such as cellulose and hemicellulose for production of useful compounds is expected to increase in the near future (Aristidou A., Pentila. M., Curr. Opin. Biotechnol, 2000, Apr., 11:2, 187-198). However, a great majority of published articles and patents, or patent applications, describe the utilization of cellulosic biomass by biocatalysts (bacteria and yeasts) for production of ethanol, which is expected to be useful as an alternative fuel. Such processes include fermentation of cellulosic biomass using different modified strains of Zymomonas mobilis (Deanda K. et al, Appl. Environ. Microbiol., 1996 December, 62:12, 4465-70; Mohagheghi A. et al, Appl. Biochem. Biotechnol., 2002, 98-100:885-98; Lawford H. G., Rousseau J. D., Appl. Biochem. Biotechnol, 2002, 98-100:429-48; PCT applications WO95/28476, WO98/50524), modified strains of Escherichia coli (Dien B. S. et al, Appl. Biochem. Biotechnol, 2000, 84-86:181-96; Nichols N. N. et al, Appl. Microbiol. Biotechnol., 2001 Jul, 56:1-2, 120-5; U.S. Pat. No. 5,000,000). Xylitol can be produced by fermentation of xylose from hemicellulosic sugars using Candida tropicalis (Walthers T. et al, Appl. Biochem. Biotechnol., 2001, 91-93:423-35). 1,2-propanediol can be produced by fermentation of arabinose, fructose, galactose, glucose, lactose, maltose, sucrose, xylose, and combination thereof using recombinant Escherichia coli strain (U.S. Pat. No. 6,303,352). Also, it has been shown that 3-dehydroshikimic acid can be obtained by fermentation of a glucose/xylose/arabinose mixture using Escherichia coli strain. The highest concentrations and yields of 3-dehydroshikimic acid were obtained when the glucose/xylose/arabinose mixture was used as the carbon source, as compared to when either xylose or glucose alone was used as a carbon source (Kai Li and J. W. Frost, Biotechnol. Prog., 1999, 15, 876-883).

It is has been reported that Escherichia coli can utilize pentoses such as L-arabinose and D-xylose (Escherichia coli and Salmonella, Second Edition, Editor in Chief: F. C. Neidhardt, ASM Press, Washington D.C., 1996). Transport of L-arabinose into the cell is performed by two inducible systems: (1) a low-affinity permease (Kₘ about 0.1 mM) encoded by araE gene, and (2) a high-affinity (Kₘ 1 to 3 μM) system encoded by the araFG operon. The araF gene encodes a periplasmic binding protein (306 amino acids) with chemotactic receptor function, and the araG locus encodes an inner membrane protein. The sugar is metabolized by a set of enzymes encoded by the araBAD operon: an isomerase (encoded by the araA gene), which reversibly converts the aldose to L-ribulose; a kinase (encoded by the araB gene), which phosphorylates the ketose to L-ribulose 5-phosphate; and L-ribulose-5-phosphate-4-epimerase (encoded by the araD gene), which catalyzes the formation of D-xylose-5-phosphate (Escherichia coli and Salmonella, Second Edition, Editor in Chief: F. C. Neidhardt, ASM Press, Washington D.C., 1996).

Most strains of E. coli grow on D-xylose, but a mutation is necessary for the K-12 strain to grow on the compound. Utilization of this pentose is through an inducible and catabolite-repressible pathway involving transport across the cytoplasmic membrane by two inducible permeases (not active on D-ribose or D-arabinose), isomerization to D-xylulose, and ATP-dependent phosphorylation of the pentulose to yield D-xylulose 5-phosphate. The high-affinity (Kₘ 0.3 to 3 μM) transport system depends on a periplasmic binding protein (37,000 Da) and is probably driven by a high-energy compound. The low-affinity (Kₘ about 170 μM) system is energized by a proton motive force. This D-xylose-proton-symport system is encoded by the xylE gene. The main gene cluster specifying D-xylose utilization is xylAB(RT). The xylA gene encodes the isomerase (54,000 Da) and xylB gene encodes the kinase (52,000 Da). The operon contains two transcriptional start points, with one of them being inserted upstream of the xylB open reading frame. Since the low-affinity permease is specified by the unlinked xylE, the xylT locus, also named as xylF (xylFGHR), probably codes for the high-affinity transport system and therefore should contain at least two genes (one for a periplasmic protein and one for an integral membrane protein) (Escherichia coli and Salmonella, Second Edition, Editor in Chief: F. C. Neidhardt, ASM Press, Washington D.C., 1996). The xylFGH genes code for xylose ABC transporters, where xylF gene encodes the putative xylose binding protein, xylG gene encodes the putative ATP-binding protein, xylH gene encodes the putative membrane component, and xylR gene encodes the xylose transcriptional activator.

Introduction of the above-mentioned E. coli genes which code for L-arabinose isomerase, L-ribulokinase, L-ribulose 5-phosphate 4-epimerase, xylose isomerase and xylulokinase, in addition to transaldolase and transketolase, allow a microbe, such as Zymomonas mobilis, to metabolize arabinose and xylose to ethanol (WO/9528476, WO98/50524). In contrast, Zymomonas genes which code for alcohol dehydrogenase (ADH) and pyruvate decarboxylase (PDH) are useful for ethanol production by Escherichia coli strains (Dien B. S. et al, Appl. Biochem. Biotechnol, 2000, 84-86:181-96; U.S. Pat. No. 5,000,000).

A process for producing L-amino acids, such as L-isoleucine, L-histidine, L-threonine and L-tryptophan, by fermentation of a mixture of glucose and pentoses, such as arabinose and xylose, was disclosed earlier by authors of the present invention (Russian patent application 2003105269).

However, at present, there are no reports describing bacteria having enhanced expression of the xylose utilization genes such as those at the xylABFGHR locus, or use of these genes for production of L-amino acids from a mixture of hexose and pentose sugars.

### SUMMARY OF THE INVENTION

An object of present invention is to enhance production of a L-amino acid producing strain, to provide a L-amino acid producing bacterium having enhanced expression of xylose utilization genes, and to provide a method for producing L-amino acids from a mixture of hexose sugars, such as glucose, and pentose sugars, such as xylose or arabinose, using the bacterium. A fermentation feedstock obtained from cellulosic biomass may be used as a carbon source for the culture medium. This aim was achieved by finding that the xylABFGHR locus cloned on a low copy vector enhances production of L-amino acids, for example, L-histidine. A microorganism is used which is capable of growth on the fermentation feedstock and is efficient in production of L-amino acids. The fermentation feedstock consists of xylose and arabinose along with glucose, as the carbon source. L-amino acid producing strains are exemplified by Escherichia coli strain. Thus the present invention has been completed.

It is an object of the present invention to provide an L-amino acid producing bacterium of the Enterobacteriaceae family which has an enhanced activity of any of the xylose utilization enzymes of.

It is a further object of the present invention to provide the bacterium described above, wherein the bacterium belongs to the genus Escherichia.

It is a further object of the present invention to provide the bacterium described above, wherein the activities of the xylose utilization enzymes are enhanced by increasing the expression amount of the xylABFGHR locus.

It is a further object of the present invention to provide the bacterium described above, wherein the activities of the xylose utilization enzymes are increased by increasing the copy number of the xylABFGHR locus or modifying an expression control sequence of the genes so that the expression of the genes are enhanced.

It is a further object of the present invention to provide the bacterium described above, wherein the copy number is increased by transforming the bacterium with a low copy vector harboring the xylABFGHR locus.

It is a further object of the present invention to provide the bacterium described above, wherein the xylABFGHR locus originates from a bacterium belonging to the genus Escherichia.

It is a further object of the present invention to provide a method for producing L-amino acids, which comprises cultivating the bacterium described above in a culture medium containing a mixture of glucose and pentose sugars, and collecting the L-amino acid from the culture medium.

It is a further object of the present invention to provide the method described above, wherein the pentose sugars are arabinose and xylose.

It is a further object of the present invention to provide the method described above, wherein the mixture of sugars is a feedstock mixture of sugars obtained from cellulosic biomass.

It is a further object of the present invention to provide the method described above, wherein the L-amino acid to be produced is L-histidine.

It is a further object of the present invention to provide the method described above, wherein the bacterium has enhanced expression of genes for L-histidine biosynthesis.

The method for producing L-amino acids includes production of L-histidine by fermentation of a mixture of glucose and pentose sugars, such as arabinose and xylose. This mixture of glucose and pentose sugars used as a fermentation feedstock can be obtained from under-utilized sources of plant biomass, such as cellulosic biomass, preferably hydrolysate of cellulose.

### BRIEF DESCRIPTION OF THE DRAWINGS

FIG. 1 shows the structure of the xylABFGHR locus on the chromosome of E. coli strain MG1655. The arrows on the diagram indicate positions of primers used in PCR.

### DETAILED DESCRIPTION OF THE INVENTION

In the present invention, “L-amino acid producing bacterium” means a bacterium, which has an ability to cause accumulation of L-amino acids in a medium, when the bacterium of the present invention is cultured in the medium. The L-amino acid producing ability may be imparted or enhanced by breeding. The term “L-amino acid producing bacterium” used herein also means a bacterium which is able to produce and cause accumulation of L-amino acids in a culture medium in amounts larger than a wild-type or parental strain, and preferably means that the microorganism is able to produce and cause accumulation in a medium of an amount not less than 0.5 g/L, more preferably not less than 1.0 g/L of target L-amino acid. “L-amino acids” include L-alanine, L-arginine, L-asparagine, L-aspartic acid, L-cysteine, L-glutamic acid, L-glutamine, L-glycine, L-histidine, L-isoleucine, L-leucine, L-lysine, L-methionine, L-phenylalanine, L-proline, L-serine, L-threonine, L-tryptophan, L-tyrosine and L-valine.

The Enterobacteriaceae family includes bacteria belonging to the genera Escherichia, Erwinia, Providencia and Serratia. The genus Escherichia is preferred.

The phrase “having enhanced activity of a xylose utilization enzyme” means that the activity of the enzyme per cell is higher than that of a non-modified strain, for example, a wild-type strain. Examples include where the number of enzyme molecules per cell increases, and where specific activity per enzyme molecule increases, and so forth. Furthermore, a wild-type strain that can act as a control includes, for example Escherichia coli K-12. As a result of enhancing the intracellular activity of a xylose utilization enzyme, L-histidine accumulation in a medium is observed.

The “xylose utilization enzymes” include enzymes of xylose transport, xylose isomerization and xylose phosphorylation, and regulatory proteins. Such enzymes include xylose isomerase, xylulokinase, xylose transporters, and xylose transcriptional activator. Xylose isomerase catalyzes the reaction of isomerization of D-xylose to D-xylulose. Xylulokinase catalyzes the reaction of phosphorylation of D-xylulose using ATP yielding D-xylulose-5-phosphate and ADP. The presence of activity of xylose utilization enzymes, such as xylose isomerase, xylulokinase, is determined by complementation of corresponding xylose isomerase-negative or xylulokinase-negative E. coli mutants, respectively.

The phrase “a bacterium belonging to the genus Escherichia” means that the bacterium is classified as the genus Escherichia according to the classification known to a person skilled in the microbiology. An example of a microorganism belonging to the genus Escherichia as used in the present invention is Escherichia coli (E. coli).

The phrase “increasing the expression amount of gene(s)” means that the expression amount of gene(s) is higher than that of a non-modified strain, for example, a wild-type strain. Examples of such modification include increasing the number of expressed gene(s) per cell, increasing the expression level of the gene(s) and so forth. The quantity of the copy number of an expressed gene is measured, for example, by restricting the chromosomal DNA followed by Southern blotting using a probe based on the gene sequence, fluorescence in situ hybridization (FISH), and the like. The level of gene expression can be measured by various methods including Northern blotting, quantitative RT-PCR, and the like. Furthermore, a wild-type strain that can act as a control includes, for example Escherichia coli K-1 2. As a result of enhancing the intracellular activity of a xylose utilization enzyme, L-histidine accumulation in a medium is observed.

Enhancing the activities of xylose utilization enzymes in a bacterial cell can be attained by increasing the expression of genes which code for said enzymes. Genes of xylose utilization include any genes derived from bacteria of Enterobacteriaceae family, as well as genes derived from other bacteria such as coryneform bacteria. Genes derived from bacteria belonging to the genus Escherichia are preferred.

The gene coding for xylose isomerase from E. coli (EC numbers 5.3.1.5) is known and has been designated xylA (nucleotide numbers 3727072 to 3728394 in the sequence of GenBank accession NC_{—}000913.1, gi:16131436). The gene coding for xylulokinase (EC numbers 2.7.1.17) is known and has been designated xylB (nucleotide numbers 3725546 to 3727000 in the sequence of GenBank accession NC_{—}000913.1, gi:16131435). The gene coding for xylose binding protein transport system is known and has been designated xylF (nucleotide numbers 3728760 to 3729752 in the sequence of GenBank accession NC_{—}000913.1, gi:16131437). The gene coding for putative ATP-binding protein of xylose transport system is known and has been designated xylG (nucleotide numbers 3729830 to 3731371 in the sequence of GenBank accession NC_{—}000913.1, gi:16131438). The gene coding for the permease component of the ABC-type xylose transport system is known and has been designated xylH gene (nucleotide numbers 3731349 to 3732530 in the sequence of GenBank accession NC_{—}000913.1, gi:16131439). The gene coding for the transcriptional regulator of the xyl operon is known and has been designated xylR (nucleotide numbers 3732608 to 3733786 in the sequence of GenBank accession NC_{—}000913.1, gi:16131440). Therefore, the above-mentioned genes can be obtained by PCR (polymerase chain reaction; refer to White, T. J. et al., Trends Genet., 5, 185 (1989)) using primers based on the nucleotide sequence of the genes.

Genes coding for xylose utilization enzymes from other microorganisms can be similarly obtained.

The xylA gene from Escherichia coli is exemplified by a DNA which encodes the following protein (A) or (B):
(A) a protein having the amino acid sequence shown in SEQ ID NO:2; or
(B) a protein having an amino acid sequence which includes deletion, substitution, insertion or addition of one or several amino acids in the amino acid sequence shown in SEQ ID NO:2, and which has an activity of xylose isomerase.

The xylB gene from Escherichia coli is exemplified by a DNA which encodes the following protein (C) or (D):
(C) a protein having the amino acid sequence shown in SEQ ID NO: 4; or
(D) a protein having an amino acid sequence which includes deletion, substitution, insertion or addition of one or several amino acids in the amino acid sequence shown in SEQ ID NO:4, and which has an activity of xylulokinase.

The xylF gene from Escherichia coli is exemplified by a DNA which encodes the following protein (E) or (F):
(E) a protein having the amino acid sequence shown in SEQ ID NO:6; or
(F) a protein having an amino acid sequence which includes deletion, substitution, insertion or addition of one or several amino acids in the amino acid sequence shown in SEQ ID NO:6, and which has activity to increase the amount of L-histidine accumulation in a medium, when the amount of protein is increased in a L-histidine producing bacterium along with the amount of proteins coded by xylAB and xylGHR genes.

The xylG gene from Escherichia coli is exemplified by a DNA which encodes the following protein (G) or (H):
(G) a protein having the amino acid sequence shown in SEQ ID NO:8; or
(H) a protein having an amino acid sequence which includes deletion, substitution, insertion or addition of one or several amino acids in the amino acid sequence shown in SEQ ID NO:8, and which has an activity to increase the amount of L-histidine accumulation in a medium, when the amount of protein is increased in a L-histidine producing bacterium along with the amount of proteins coded by xylAB and xylFHR genes.

The xylH gene from Escherichia coli is exemplified by a DNA which encodes the following protein (I) or (J):
(I) a protein having the amino acid sequence shown in SEQ ID NO:10;

(J) a protein having an amino acid sequence including deletion, substitution, insertion or addition of one or several amino acids in the amino acid sequence shown in SEQ ID NO: 10, and which has an activity to increase the amount of L-histidine accumulation in a medium, when the amount of protein is increased in a L-histidine producing bacterium along with the amount of proteins coded by xylAB and xylFGR genes.

The xylR gene from Escherichia coli is exemplified by a DNA which encodes the following protein (K) or (L):
(K) a protein having the amino acid sequence shown in SEQ ID NO:12;

(L) a protein having an amino acid sequence including deletion, substitution, insertion or addition of one or several amino acids in the amino acid sequence shown in SEQ ID NO:12, and which has an activity to increase the amount of L-histidine accumulation in a medium, when the amount of protein is increased in a L-histidine producing bacterium along with the amount of proteins coded by xylAB and xylFGH genes.

The DNA coding for xylose isomerase includes a DNA coding for the protein which includes deletion, substitution, insertion or addition of one or several amino acids in one or more positions on the protein (A) as long as the activity of the protein is not lost. Although the number of “several” amino acids differs depending on the position or the type of amino acid residues in the three-dimensional structure of the protein, it may be 2 to 50, preferably 2 to 20, and more preferably 2 to 10 for the protein (A). This is because some amino acids have high homology to one another and substitution of such an amino acid does not greatly affect the three dimensional structure of the protein and its activity. Therefore, the protein (B) may have homology of not less than 30 to 50%, preferably 50 to 70%, more preferably 70-90%, still more preferably more then 90% and most preferably more than 95% with respect to the entire amino acid sequence for xylose isomerase, and which has the activity of xylose isomerase. The same approach and homology determination can be applied to other proteins (C), (E), (G), (I) and (K).

To evaluate the degree of protein or DNA homology, several calculation methods such as BLAST search, FASTA search and CrustalW, can be used.

BLAST (Basic Local Alignment Search Tool) is the heuristic search algorithm employed by the programs blastp, blastn, blastx, megablast, tblastn, and tblastx; these programs ascribe significance to their findings using the statistical methods of Karlin, Samuel and Stephen F. Altschul (“Methods for assessing the statistical significance of molecular sequence features by using general scoring schemes”. Proc. Natl. Acad. Sci. USA, 1990, 87:2264-68; “Applications and statistics for multiple high-scoring segments in molecular sequences”. Proc. Natl. Acad. Sci. USA, 1993, 90:5873-7). FASTA search method described by W. R. Pearson (“Rapid and Sensitive Sequence Comparison with FASTP and FASTA”, Methods in Enzymology, 1990 183:63-98). Clustal W method described by Thompson J. D., Higgins D. G. and Gibson T. J. (“CLUSTAL W: improving the sensitivity of progressive multiple sequence alignment through sequence weighting, position-specific gap penalties and weight matrix choice”, Nucleic Acids Res. 1994, 22:4673-4680).

Changes to the protein defined in (A) such as those described above are typically conservative changes so as to maintain the activity of the protein. Substitution changes include those in which at least one residue in the amino acid sequence has been removed and a different residue inserted in its place. Examples of amino acids which may be substituted for an original amino acid in the above protein and which are regarded as conservative substitutions include: Ala substituted with ser or thr; arg substituted with gln, his, orlys; asn substituted with glu, gin, lys, his, asp; asp substituted with asn, glu, or gin; cys substituted with ser or ala; gin substituted with asn, glu, lys, his, asp, or arg; glu substituted with asn, gin, lys, or asp; gly substituted with pro; his substituted with asn, lys, gin, arg, tyr; ile substituted with leu, met, yal, phe; leu substituted with ile, met, val, phe; lys substituted with asn, glu, gin, his, arg; met substituted with ile, leu, val, phe; phe substituted with trp, tyr, met, ile, or leu; ser substituted with thr, ala; thr substituted with ser or ala; trp substituted with phe, tyr; tyr substituted with his, phe, or trp; and val substituted with met, ile, leu.

The DNA coding for substantially the same protein as the protein defined in (A) may be obtained by, for example, modification of the nucleotide sequence coding for the protein defined in (A) using site-directed mutagenesis so that one or more amino acid residue will be deleted, substituted, inserted or added. Such modified DNA can be obtained by conventional methods using treatments with reagents and conditions generating mutations. Such treatments include treating the DNA coding for proteins of present invention with hydroxylamine or treating the bacterium harboring the DNA with UV irradiation or reagents such as N-methyl-N′-nitro-N-nitrosoguanidine or nitrous acid.

The DNA coding for the xylose isomerase includes variants which can be found in the different strains of bacteria belonging to the genus Escherichia due to natural diversity. The DNA coding for such variants can be obtained by isolating the DNA which hybridizes with the xylA gene or a part of the gene under the stringent conditions, and which codes for the protein having an activity of xylose isomerase. The phrase “stringent conditions” referred to herein include conditions under which a so-called specific hybrid is formed, and non-specific hybrid is not formed. For example, the stringent conditions include conditions under which DNAs having high homology, for instance DNAs having homology no less than 70%, preferably no less than 80%, more preferably no less than 90%, most preferably no less than 95% to each other, are hybridized. Alternatively, the stringent conditions are exemplified by conditions which comprise ordinary conditions of washing in Southern hybridization, e.g., 60° C., 1×SSC, 0.1% SDS, preferably 0.1×SSC, 0.1% SDS. Duration of the washing procedure depends on the type of membrane used for blotting and, as a rule, what is recommended by manufacturer. For example, recommended duration of washing the Hybond™ N+nylon membrane (Amersham) under stringent conditions is 15 minutes. Preferably, washing may be performed 2 to 3 times. A partial sequence of the nucleotide sequence of SEQ ID NO: 1 can also be used as a probe for DNA that codes for variants and hybridizes with xylA gene. Such a probe may be prepared by PCR using oligonucleotides produced based on the nucleotide sequence of SEQ ID NO: 1 as primers, and a DNA fragment containing the nucleotide sequence of SEQ ID NO: 1 as a template. When a DNA fragment in a length of about 300 bp is used as the probe, the conditions of washing for the hybridization can be, for example, 50° C., 2×SSC, and 0.1%'sDS.

DNAs coding for substantially the same proteins as the other enzymes of xylose utilization can be obtained by methods which are similar to those used to obtain xylose isomerase, as described above.

Transformation of a bacterium with a DNA coding for a protein means introduction of the DNA into a bacterium cell, for example, by conventional methods to increase expression of the gene coding for the protein of present invention and to enhance the activity of the protein in the bacterial cell.

The bacterium of the present invention also includes one where the activity of the protein of the present invention is enhanced by transformation of said bacterium with a DNA coding for a protein as defined in (A) or (B), (C) or (D), (E) or (F), (G) or (H), (I) or (J), and (K) or (L), or by alteration of expression regulation sequence of said DNA on the chromosome of the bacterium.

A method of the enhancing gene expression includes increasing the gene copy number. Introduction of a gene into a vector that is able to function in a bacterium belonging to the genus Escherichia increases copy number of the gene. For such purposes multi-copy vectors can be preferably used. Preferably, low copy vectors are used. The low-copy vector is exemplified by pSC101, pMW118, pMW119 and the like. The term “low copy vector” is used for vectors which have a copy number of up to 5 copies per cell. Methods of transformation include any method known to those with skill in the art. For example, a method of treating recipient cells with calcium chloride so as to increase permeability of the cells to DNA has been reported for Escherichia coli K-12 (Mandel, M. and Higa, A., J. Mol. Biol., 53, 159 (1970)) and may be used.

Enhancement of gene expression may also be achieved by introduction of multiple copies of the gene into a bacterial chromosome by, for example, a method of homologous recombination, Mu integration or the like. For example, one round of Mu integration allows introduction into a bacterial chromosome of up to 3 copies of the gene.

On the other hand, the enhancement of gene expression can be achieved by placing the DNA of the present invention under the control of a more potent promoter instead of the native promoter. The strength of a promoter is defined by the frequency of acts of RNA synthesis initiation. Methods for evaluation of the strength of a promoter and examples of potent promoters are described by Deuschle, U., Kammerer, W., Gentz, R., Bujard, H. (Promoters in Escherichia coli: a hierarchy of in vivo strength indicates alternate structures. EMBO J. 1986, 5, 2987-2994). For example, PR promoter is known as a potent constitutive promoter. Other known potent promoters are PL promoter, lac promoter, trp promoter, trc promoter, of lambda phage and the like.

The enhancement of translation can be achieved by introducing a more efficient Shine-Dalgarno sequence (SD sequence) into the DNA of the present invention instead of the native SD sequence. The SD sequence is a region upstream of the start codon of the mRNA which interacts with the 16S RNA of the ribosome (Shine J. and Dalgarno L., Proc. Natl. Acad. Sci. USA, 1974, 71, 4, 1342-6).

Use of a more potent promoter can be combined with the multiplication of gene copies method.

Alternatively, a promoter can be enhanced by, for example, introducing a mutation into the promoter to increase a transcription level of a gene located downstream of the promoter. Furthermore, it is known that substitution of several nucleotides in a spacer between the ribosome binding site (RBS) and start codon, and particularly, the sequences immediately upstream of the start codon profoundly affect the mRNA translatability. For example, a 20-fold range in the expression levels was found, depending on the nature of the three nucleotides preceding the start codon (Gold et al., Annu. Rev. Microbiol., 35, 365-403, 1981; Hui et al., EMBO J., 3, 623-629, 1984).

Methods for preparation of chromosomal DNA, hybridization, PCR, preparation of plasmid DNA, digestion. and ligation of DNA, transformation, selection of an oligonucleotide as a primer and the like can be ordinary methods well known to one skilled in the art. These methods are described in Sambrook, J., and Russell D., “Molecular Cloning A Laboratory Manual, Third Edition”, Cold Spring Harbor Laboratory Press (2001), and the like.

The bacterium of the present invention can be obtained by introduction of the aforementioned DNAs into a bacterium inherently having an ability to produce L-histidine. Alternatively, the bacterium of present invention can be obtained by imparting an ability to produce L-histidine to the bacterium already harboring the DNAs.

Examples of L-amino acid producing bacteria belonging to the genus Escherichia are described below.

L-histidine producing bacteria

Examples of bacteria belonging to the genus Escherichia having L-histidine producing ability include L-histidine producing bacterium strains belonging to the genus Escherichia, such as E. coli strain 24 (VKPM B-5945, RU2003677); E. coli strain 80 (VKPM B-7270, RU2119536); E. coli strains NRRL B- 12116-B12121 (U.S. Pat. No. 4,388,405); E. coli strains H-9342 (FERM BP-6675) and H-9343 (FERM BP-6676) (U.S. Pat. No. 6,344,347); E. coli strain H-9341 (FERM BP-6674) (EP1085087); E. coli strain AI80/pFM201 (U.S. Pat. No. 6,258,554) and the like.

The above-mentioned L-amino acid producing strains may be further modified for enhancement of the pentose assimilation rate or for enhancement of the L-amino acid biosynthetic ability by the wide scope of methods well known to the person skilled in the art.

The utilization rate for pentose sugars can be further enhanced by amplification of the pentose assimilation genes, araFG and araBAD genes for arabinose, or by mutations in the glucose assimilation systems (PTS and non-PTS), such asptsG mutations (Nichols N. N. et al, Appl. Microbiol. Biotechnol., 2001, July 56:1-2, 120-5).

The biosynthetic ability of the L-amino acid producing bacterium may be further improved by enhancing the expression of one or more genes which are involved in L-amino acid biosynthesis. Such genes are exemplified by the histidine operon, which preferably includes the hisG gene encoding ATP phosphoribosyl transferase of which feedback inhibition by L-histidine is desensitized (Russian patents 2003677 and 2119536), for L-histidine producing bacteria.

The process of the present invention includes a process for producing an L-amino acid comprising the steps of cultivating the L-amino acid producing bacterium in a culture medium, allowing the L-amino acid to accumulate in the culture medium, and collecting the L-amino acid from the culture medium, wherein the culture medium contains a mixture of glucose and pentose sugars. Also, the method of present invention includes a method for producing L-histidine comprising the steps of cultivating the L-histidine producing bacterium of the present invention in a culture medium, allowing L-histidine to accumulate in the culture medium, and collecting L-histidine from the culture medium, wherein the culture medium contains a mixture of glucose and pentose sugars.

The mixture of pentose sugars, such as xylose and arabinose, along with hexose sugar, such as glucose, can be obtained from under-utilized sources of biomass. Glucose, xylose, arabinose and other carbohydrates are liberated from plant biomass by steam and/or concentrated acid hydrolysis, dilute acid hydrolysis, hydrolysis using enzymes, such as cellulase, or alkali treatment. When the substrate is cellulosic material, the cellulose may be hydrolyzed to sugars simultaneously or separately and also fermented to L-amino acid. Since hemicellulose is generally easier to hydrolyze to sugars than cellulose, it is preferable to prehydrolyze the cellulosic material, separate the pentoses and then hydrolyze the cellulose by treatment with steam, acid, alkali, cellulases or combinations thereof to form glucose.

A mixture consisting of different ratios of glucose/xylose/arabinose was used in this study to approximate the composition of feedstock mixture of glucose and pentoses, which could potentially be derived from plant hydrolysates (see Example section).

In the present invention, the cultivation, the collection and purification of L-amino acid from the medium and the like may be performed in a manner similar to a conventional fermentation method wherein an amino acid is produced using a microorganism. The medium used for culture may be either a synthetic medium or a natural medium, so long as the medium includes a carbon source and a nitrogen source and minerals and, if necessary, appropriate amounts of nutrients which the microorganism requires for growth.

The carbon source may include various carbohydrates such as glucose, sucrose, arabinose, xylose and other pentose and hexose sugars, which the L-amino acid producing bacterium could utilize as a carbon source. Glucose, xylose, arabinose and other carbohydrates may be a part of feedstock mixture of sugars obtained from cellulosic biomass.

Pentose sugars suitable for fermentation in the present invention include, but are not limited to xylose and arabinose.

As the nitrogen source, various ammonium salts such as ammonia and ammonium sulfate, other nitrogen compounds such as amines, a natural nitrogen source such as peptone, soybean-hydrolysate and digested fermentative microorganism are used. As minerals, potassium monophosphate, magnesium sulfate, sodium chloride, ferrous sulfate, manganese sulfate, calcium chloride, and the like are used. Additional nutrients can be added to the medium if necessary. For instance, if the microorganism requires proline for growth (proline auxotrophy) a sufficient amount of proline can be added to the medium for cultivation.

Preferably, the cultivation is performed under aerobic conditions such as a shaking culture, and a stirring culture with aeration, at a temperature of 20 to 40° C., preferably 30 to 38° C. The pH of the culture is usually between 5 and 9, preferably between 6.5 and 7.2. The pH of the culture can be adjusted with ammonia, calcium carbonate, various acids, various bases, and buffers. Usually, a 1 to 5-day cultivation leads to the accumulation of the target L-amino acid in the liquid medium.

After cultivation, solids such as cells can be removed from the liquid medium by centrifugation or membrane filtration, and then the target L-amino acid can be collected and purified by ion-exchange, concentration and crystallization methods.

### EXAMPLES

The present invention will be more concretely explained below with reference to the following non-limiting examples.

### Example 1

### Cloning the xylABFGHR Locus from the Chromosome of E. coli Strain MG1655

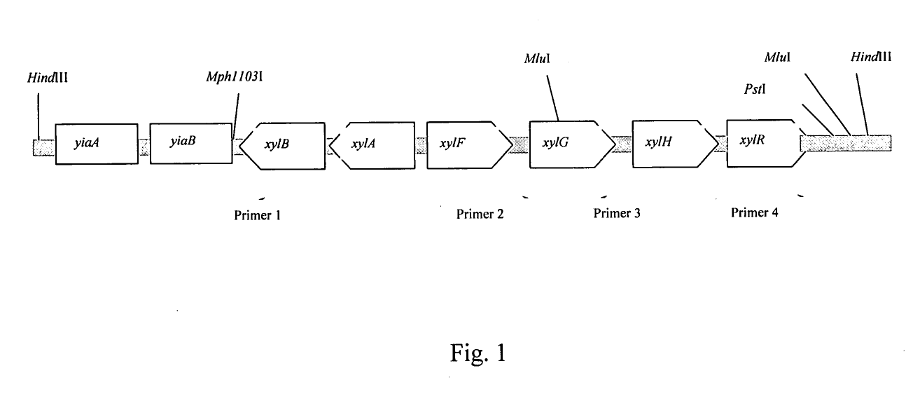

Based on genome analysis of E. coli strain MG1655, the genes xylABFGHR can be cloned as a single HindIII fragment (13.1 kb) of 556 HindIII chromosomal fragments in total (FIG. 1). For that purpose, a gene library was constructed using vector pUC 19, which is capable of surviving in E. coli with insertions of that size.

To construct such a library, chromosomal DNA of MG1655 was digested with HindIII restrictases and the pUC 19 vector was digested with XbaI restrictase. The strain MG1655 (ATCC47076, ATCC700926) can be obtained from American Type Culture Collection (10801 University Boulevard, Manassas, Va., 20110-2209, U.S.A.)

Sticky ends in both DNA preparations were subsequently filled by Klenow fragment so as to prevent self-ligation (two bases filling). After the ligation procedure a pool of recombinant pUC19 plasmids was obtained. The size of the library is more then 200 thousand clones. The gene library was analyzed by PCR using primers complementary to the plasmid sequence and primers complementary to the cloning chromosomal fragment. DNA fragments with appropriate molecular weights were not found among the PCR products, which was interpreted to mean that the fragment corresponding to the xylABFGHR operon was missing from the constructed library. This result may be due to the negative influence of the malS gene, and the yiaA and yiaB ORFs (with unknown function), which are also present in the HindIII fragment of interest. Another possible reason for negative selection is the large size of the Xyl-locus. To overcome this problem, new gene libraries were constructed based on a modified pUC 19 plasmid. The main approach is to clone Xyl-locus as a set of fragments without of the adjacent malS gene and yiaa and yiaB ORFs.

For that purpose, a polylinker of plasmid pUC 19 was modified by inserting a synthetic DNA fragment containing MluI restriction site. Two gene libraries were constructed in the modified pUC19 cloning vector. The first library was created by digestion of the chromosomal DNA of strain MG1655 and the modified pUC 19 with HindIII and MluI restrictases followed by ligation. The library volume was more than 4,000 clones. The gene library was analyzed by PCR using primers complementary to the plasmid sequence, and primers 1 (SEQ ID NO:13) and 2 (SEQ ID NO:14) which are complementary to the fragment xylABFG of the xyl locus. The expected DNA fragments with appropriate molecular weights were found among the PCR products. The next step was to saturate the gene library with a fragment of interest. To this end, DNA from the original gene library was digested by endonucleases, restriction sites of which do not exist in the fragment of interest. There are Eco 147I, KpnI, MlsI, Bst11071. The frequency of the plasmid of interest in the enriched library was 1/800 clones. The enriched library was analyzed by PCR as described above. After five sequential enrichments of the library the cell population, only ten clones containing xylABFG genes were found. The resulting plasmid containing HindIII—MluI DNA fragment with genes xylABFG was designated as pUC19/xylA-G. Then the HindIII-Mph1103I fragment containing theyiaA andyiaB ORFs was eliminated from plasmid pUC19/xylA-G; sticky ends were blunted by Klenow fragment and a synthetic linker containing an EcoRI restriction site was inserted by ligation. Thus, the plasmid pUC19/xylA-G-2 was obtained. Then, the resulting pUC19/xylA-G-2 plasmid was cut by an Ehel restrictase; sticky ends were blunted by Klenow fragment and synthetic linker containing HindIII restriction site was inserted by ligation. Thus the pUC19/xylA-G-3 plasmid was obtained. A HindIII restriction site was inserted with the remaining DNA fragment containing xylHR genes, resulting in the complete xyl locus.

The second library was created by digestion of the chromosomal DNA from strain MG1655 and a modified pUC19 with PstI and MluI restrictases, followed by ligation. The library volume was more than 6,000 clones. The gene library was analyzed by PCR using primers complementary to the plasmid sequence and primers 3 (SEQ ID NO: 15) and 4 (SEQ ID NO: 16), which are complementary to the cloning chromosomal fragment. DNA fragments with appropriate molecular weights were found among the PCR products. The next step was a sequential subdivision of the gene library on cell population with known size, accompanied by PCR analysis. After seven sequential subdivision of library the cell population containing genes xylHR contained only ten clones. Among this population, a fragment DNA of interest was found by restriction analysis. The resulting plasmid containing Pstl—MluI DNA fragment with xylHR genes was designated as pUC19/xylHR. Then, HindIII-MluI DNA fragment from plasmid pUC19/xylHR was ligated to the pUC19/xylA-G-3 plasmid, which had been previously treated with HindIII and MluI restrictases. Finally, the complete xyl locus of strain MG1655 was obtained. The resulting multicopy plasmid containing the complete xylABFGHR locus was designated pUC19/xylA-R.

Then HindIII-EcoRI DNA fragment from the pUC19/xylA-R plasmid was recloned into the low copy vector pMW119mod, which had been previously digested with HindIII and EcoRI restrictases, resulting in the low copy plasmid pMW119mod-xylA-R which contained the complete xylABFGHR locus. The low copy vector pMW119mod was obtained from the commercially available pMW119 vector by elimination of the PvuII-PvuII fragment. This fragment contains the multi-cloning site and was a major part of the lacZ gene. The lacZ gene contains sites for laci repressor followed by insertion of synthetic linker containing EcoRI and HindIII sites, which are necessary for insertion of the xylABFGHR locus from the pUC 19/xylA-R plasmid.

### Example 2

### Production of L-Histidine by L-Histidine Producing Bacterium from Fermentation of a Mixture of Glucose and Pentoses

L-histidine producing E. coli strain 80 was used as a strain for production of L-histidine by fermentation of a mixture of glucose and pentoses. E. coli strain 80 (VKPM B-7270) is described in detail in Russian patent RU2119536 and has been deposited in the Russian National Collection of Industrial Microorganisms (Russia, 113545 Moscow, 1st Dorozhny proezd, 1) on Oct. 15, 1999 under accession number VRPM B-7270. Then, it was transferred to an international deposit under the provisions of the Budapest Treaty on Jul. 12, 2004. Transformation of strain 80 with the pMW119mod-xylA-R plasmid was performed by ordinary methods, yielding strain 80/pMW119mod-xylA-R.

To obtain the seed culture, both strains 80 and 80/pMW119mod-xylA-R were grown on a rotary shaker (250 rpm) at 27° C. for 6 hours in 40 ml test tubes (Ø 18 mm) containing 2 ml of L-broth with 1 g/l of streptomycin and. For the strain 80/pMW119mod-xylA-R, 100 mg/l ampicillin was additionally added. Then, 2 ml (5%) of seed material was inoculated into the fermentation medium. Fermentation was carried out on a rotary shaker (250 rpm) at 27° C. for 65 hours in 40 ml test tubes containing 2 ml of fermentation medium.

After cultivation, the amount of L-histidine which had accumulated in the culture medium was determined by paper chromatography. The composition of the mobile phase is the following: butanol : acetate : water=4:1:1 (v/v). A solution (0.5%) of ninhydrin in acetone was used as a visualizing reagent. The results are presented in Table 2.

The Composition of the Fermentation Medium (g/l):

| | |
|---|---|
| Carbohydrates (total) | 100.0 |
| Mameno | 0.2 |
| (soybean hydrolysate) | of TN (total nitrogen) |
| L-proline | 0.8 |
| (NH₄)₂SO₄ | 25.0 |
| K₂HPO₄ | 2.0 |
| MgSO₄.7H₂O | 1.0 |
| FeSO₄.7H₂O | 0.01 |
| MnSO₄.5H₂O | 0.01 |
| Thiamine HCI | 0.001 |
| Betaine | 2.0 |
| CaCO₃ | 6.0 |
| Streptomycin | 1.0 |

Carbohydrates (glucose, arabinose, xylose), L-proline, betaine and magnesium sulfate are sterilized separately. CaCO₃ dry-heat are sterilized at 110° C. for 30 min. pH is adjusted to 6.0 by KOH before sterilization.

TABLE 2

| | | | | | Glucose/ | | | | Glucose/ | |
|---|---|---|---|---|---|---|---|---|---|---|
| | Glucose | | Xylose | | xylose 1:1 | | Arabinose | | arabinose 1:1 | |
| | | His, | | His, | | His, | | His, | | His, |
| Strain | OD₄₅₀ | g/l | OD₄₅₀ | g/l | OD₄₅₀ | g/l | OD₄₅₀ | g/l | OD₄₅₀ | g/l |
| 80 | 43 | 8.9 | No | 0.4 | 39 | 3.2 | 37 | 10.3 | 40 | 8.7 |
| | | | growth | | | | | | | |
| 80/pMW119mod- | 39 | 9.3 | 50 | 9.6 | 39 | 9.9 | 36 | 10.5 | 40 | 9.1. |
| xylA-R | | | | | | | | | | |

As can be seen from Table 2, increased expression of the xylABFGHR locus improved productivity of the L-histidine producing E. coli strain 80 cultured in the medium containing xylose.

While the invention has been described in detail with reference to preferred embodiments thereof, it will be apparent to one skilled in the art that various changes can be made, and equivalents employed, without departing from the scope of the invention. Each of the aforementioned documents, including Russian Patent Appln. No. 2004107548 filed on Mar. 16, 2004 and U.S. patent application Ser. No. 60/610,545 filed on Sep. 17, 2004, is incorporated by reference herein in its entirety.

1. An L-amino acid producing bacterium of the Enterobacteriaceae family, said bacterium having enhanced activities of any of the xylose utilization enzymes.

2. The bacterium according to claim 1 wherein the bacterium belongs to the genus Escherichia.

3. The bacterium according to claim 1 wherein the activities of xylose utilization enzymes are enhanced by increasing the expression amount of the xylABFGHR locus.

4. The bacterium according to claim 3, wherein the activities of xylose utilization enzymes are enhanced by increasing the copy number of the xylABFGHR locus or modifying an expression control sequence so that the expression of the genes are enhanced.

5. The bacterium according to claim 4, wherein the copy number is increased by transforming the bacterium with a low-copy vector harboring the xylABFGHR locus.

6. The bacterium according to any of claim 3, wherein the xylABFGHR locus originates from a bacterium belonging to the genus Escherichia.

7. A method for producing an L-amino acid, the method comprising cultivating the bacterium according to any of claims 1 in a culture medium containing a mixture of glucose and pentose sugars, and collecting the L-amino acid from the culture medium.

8. The method according to claim 7, wherein the pentose sugars are arabinose and xylose.

9. The method according to claim 8, wherein the mixture of sugars is a feedstock mixture of sugars obtained from cellulosic biomass.

10. The method according to claim 9, wherein the L-amino acid to be produced is L-histidine.

11. The method according to claim 10, wherein the bacterium has enhanced expression of genes for L-histidine biosynthesis.


SEQUENCE LISTING (16 sequences)

SEQ ID NO: 1 - DNA, 1323 bp, Escherichia coli
  CDS (1)..(1323)
atgcaagcctattttgaccagctcgatcgcgttcgttatgaaggctcaaaatcctcaaacccgttagcattccgtcactacaatcccgacgaactggtgttgggtaagcgtatggaagagcacttgcgttttgccgcctgctactggcacaccttctgctggaacggggcggatatgtttggtgtgggggcgtttaatcgtccgtggcagcagcctggtgaggcactggcgttggcgaagcgtaaagcagatgtcgcatttgagtttttccacaagttacatgtgccattttattgcttccacgatgtggatgtttcccctgagggcgcgtcgttaaaagagtacatcaataattttgcgcaaatggttgatgtcctggcaggcaagcaagaagagagcggcgtgaagctgctgtggggaacggccaactgctttacaaaccctcgctacggcgcgggtgcggcgacgaacccagatcctgaagtcttcagctgggcggcaacgcaagttgttacagcgatggaagcaacccataaattgggcggtgaaaactatgtcctgtggggcggtcgtgaaggttacgaaacgctgttaaataccgacttgcgtcaggagcgtgaacaactgggccgctttatgcagatggtggttgagcataaacataaaatcggtttccagggcacgttgcttatcgaaccgaaaccgcaagaaccgaccaaacatcaatatgattacgatgccgcgacggtctatggcttcctgaaacagtttggtctggaaaaagagattaaactgaacattgaagctaaccacgcgacgctggcaggtcactctttccatcatgaaatagccaccgccattgcgcttggcctgttcggttctgtcgacgccaaccgtggcgatgcgcaactgggctgggacaccgaccagttcccgaacagtgtggaagagaatgcgctggtgatgtatgaaattctcaaagcaggcggtttcaccaccggtggtctgaacttcgatgccaaagtacgtcgtcaaagtactgataaatatgatctgttttacggtcatatcggcgcgatggatacgatggcactggcgctgaaaattgcagcgcgcatgattgaagatggcgagctggataaacgcatcgcgcagcgttattccggctggaatagcgaattgggccagcaaatcctgaaaggccaaatgtcactggcagatttagccaaatatgctcaggaacatcatttgtctccggtgcatcagagtggtcgccaggaacaactggaaaatctggtaaaccattatctgttcgacaaataa

SEQ ID NO: 2 - protein, 440 aa, Escherichia coli
MQAYFDQLDRVRYEGSKSSNPLAFRHYNPDELVLGKRMEEHLRFAACYWHTFCWNGADMFGVGAFNRPWQQPGEALALAKRKADVAFEFFHKLHVPFYCFHDVDVSPEGASLKEYINNFAQMVDVLAGKQEESGVKLLWGTANCFTNPRYGAGAATNPDPEVFSWAATQVVTAMEATHKLGGENYVLWGGREGYETLLNTDLRQEREQLGRFMQMVVEHKHKIGFQGTLLIEPKPQEPTKHQYDYDAATVYGFLKQFGLEKEIKLNIEANHATLAGHSFHHEIATAIALGLFGSVDANRGDAQLGWDTDQFPNSVEENALVMYEILKAGGFTTGGLNFDAKVRRQSTDKYDLFYGHIGAMDTMALALKIAARMIEDGELDKRIAQRYSGWNSELGQQILKGQMSLADLAKYAQEHHLSPVHQSGRQEQLENLVNHYLFDK

SEQ ID NO: 3 - DNA, 1455 bp, Escherichia coli
  CDS (1)..(1455)
atgtatatcgggatagatcttggcacctcgggcgtaaaagttattttgctcaacgagcagggtgaggtggttgctgcgcaaacggaaaagctgaccgtttcgcgcccgcatccactctggtcggaacaagacccggaacagtggtggcaggcaactgatcgcgcaatgaaagctctgggcgatcagcattctctgcaggacgttaaagcattgggtattgccggccagatgcacggagcaaccttgctggatgctcagcaacgggtgttacgccctgccattttgtggaacgacgggcgctgtgcgcaagagtgcactttgctggaagcgcgagttccgcaatcgcgggtgattaccggcaacctgatgatgcccggatttactgcgcctaaattgctatgggttcagcggcatgagccggagatattccgtcaaatcgacaaagtattattaccgaaagattacttgcgtctgcgtatgacgggggagtttgccagcgatatgtctgacgcagctggcaccatgtggctggatgtcgcaaagcgtgactggagtgacgtcatgctgcaggcttgcgacttatctcgtgaccagatgcccgcattatacgaaggcagcgaaattactggtgctttgttacctgaagttgcgaaagcgtggggtatggcgacggtgccagttgtcgcaggcggtggcgacaatgcagctggtgcagttggtgtgggaatggttgatgctaatcaggcaatgttatcgctggggacgtcgggggtctattttgctgtcagcgaagggttcttaagcaagccagaaagcgccgtacatagcttttgccatgcgctaccgcaacgttggcatttaatgtctgtgatgctgagtgcagcgtcgtgtctggattgggccgcgaaattaaccggcctgagcaatgtcccagctttaatcgctgcagctcaacaggctgatgaaagtgccgagccagtttggtttctgccttatctttccggcgagcgtacgccacacaataatccccaggcgaagggggttttctttggtttgactcatcaacatggccccaatgaactggcgcgagcagtgctggaaggcgtgggttatgcgctggcagatggcatggatgtcgtgcatgcctgcggtattaaaccgcaaagtgttacgttgattgggggcggggcgcgtagtgagtactggcgtcagatgctggcggatatcagcggtcagcagctcgattaccgtacggggggggatgtggggccagcactgggcgcagcaaggctggcgcagatcgcggcgaatccagagaaatcgctcattgaattgttgccgcaactaccgttagaacagtcgcatctaccagatgcgcagcgttatgccgcttatcagccacgacgagaaacgttccgtcgcctctatcagcaacttctgccattaatggcgtaa

SEQ ID NO: 4 - protein, 484 aa, Escherichia coli
MYIGIDLGTSGVKVILLNEQGEVVAAQTEKLTVSRPHPLWSEQDPEQWWQATDRAMKALGDQHSLQDVKALGIAGQMHGATLLDAQQRVLRPAILWNDGRCAQECTLLEARVPQSRVITGNLMMPGFTAPKLLWVQRHEPEIFRQIDKVLLPKDYLRLRMTGEFASDMSDAAGTMWLDVAKRDWSDVMLQACDLSRDQMPALYEGSEITGALLPEVAKAWGMATVPVVAGGGDNAAGAVGVGMVDANQAMLSLGTSGVYFAVSEGFLSKPESAVHSFCHALPQRWHLMSVMLSAASCLDWAAKLTGLSNVPALIAAAQQADESAEPVWFLPYLSGERTPHNNPQAKGVFFGLTHQHGPNELARAVLEGVGYALADGMDVVHACGIKPQSVTLIGGGARSEYWRQMLADISGQQLDYRTGGDVGPALGAARLAQIAANPEKSLIELLPQLPLEQSHLPDAQRYAAYQPRRETFRRLYQQLLPLMA

SEQ ID NO: 5 - DNA, 993 bp, Escherichia coli
  CDS (1)..(993)
atgaaaataaagaacattctactcaccctttgcacctcactcctgcttaccaacgttgctgcacacgccaaagaagtcaaaataggtatggcgattgatgatctccgtcttgaacgctggcaaaaagatcgagatatctttgtgaaaaaggcagaatctctcggcgcgaaagtatttgtacagtctgcaaatggcaatgaagaaacacaaatgtcgcagattgaaaacatgataaaccggggtgtcgatgttcttgtcattattccgtataacggtcaggtattaagtaacgttgtaaaagaagccaaacaagaaggcattaaagtattagcttacgaccgtatgattaacgatgcggatatcgatttttatatttctttcgataacgaaaaagtcggtgaactgcaggcaaaagccctggtcgatattgttccgcaaggtaattacttcctgatgggcggctcgccggtagataacaacgccaagctgttccgcgccggacaaatgaaagtgttaaaaccttacgttgattccggaaaaattaaagtcgttggtgaccaatgggttgatggctggttaccggaaaacgcattgaaaattatggaaaacgcgctaaccgccaataataacaaaattgatgctgtagttgcctcaaacgatgccaccgcaggtggggcaattcaggcattaagcgcgcaaggtttatcagggaaagtagcaatctccggccaggatgcggatctcgcaggtattaaacgtattgctgccggtacgcaaactatgacggtgtataaacctattacgttgttggcaaatactgccgcagaaattgccgttgagttgggcaatggtcaggaaccaaaagcagataccacactgaataatggcctgaaagatgtcccctcccgcctcctgacaccgatcgatgtgaataaaaacaacatcaaagatacggtaattaaagacggattccacaaagagagcgagctgtaa

SEQ ID NO: 6 - protein, 330 aa, Escherichia coli
MKIKNILLTLCTSLLLTNVAAHAKEVKIGMAIDDLRLERWQKDRDIFVKKAESLGAKVFVQSANGNEETQMSQIENMINRGVDVLVIIPYNGQVLSNVVKEAKQEGIKVLAYDRMINDADIDFYISFDNEKVGELQAKALVDIVPQGNYFLMGGSPVDNNAKLFRAGQMKVLKPYVDSGKIKVVGDQWVDGWLPENALKIMENALTANNNKIDAVVASNDATAGGAIQALSAQGLSGKVAISGQDADLAGIKRIAAGTQTMTVYKPITLLANTAAEIAVELGNGQEPKADTTLNNGLKDVPSRLLTPIDVNKNNIKDTVIKDGFHKESEL

SEQ ID NO: 7 - DNA, 1542 bp, Escherichia coli
  CDS (1)..(1542)
atgccttatctacttgaaatgaagaacattaccaaaaccttcggcagtgtgaaggcgattgataacgtctgcttgcggttgaatgctggcgaaatcgtctcactttgtggggaaaatgggtctggtaaatcaacgctgatgaaagtgctgtgtggtatttatccccatggctcctacgaaggcgaaattatttttgcgggagaagagattcaggcgagtcacatccgcgataccgaacgcaaaggtatcgccatcattcatcaggaattggccctggtgaaagaattgaccgtgctggaaaatatcttcctgggtaacgaaataacccacaatggcattatggattatgacctgatgacgctacgctgtcagaagctgctcgcacaggtcagtttatccatttcacctgatacccgcgttggcgatttagggcttgggcaacaacaactggttgaaattgccaaggcacttaataaacaggtgcgcttgttaattctcgatgaaccgacagcctcattaactgagcaggaaacgtcgattttactggatattattcgcgatctacaacagcacggtatcgcctgtatttatatttcgcacaaactcaacgaagtcaaagcgatttccgatacgatttgcgttattcgcgacggacagcacattggtacgcgtgatgctgccggaatgagtgaagacgatattatcaccatgatggtcgggcgagagttaaccgcgctttaccctaatgaaccacataccaccggagatgaaatattacgtattgaacatctgacggcatggcatccggttaatcgtcatattaaacgagttaatgatgtctcgttttccctgaaacgtggcgaaatattgggtattgccggactcgttggtgccggacgtaccgagaccattcagtgcctgtttggtgtgtggcccggacaatgggaaggaaaaatttatattgatggcaaacaggtagatattcgtaactgtcagcaagccatcgcccaggggattgcgatggtccccgaagacagaaagcgcgacggcatcgttccggtaatggcggttggtaaaaatattaccctcgccgcactcaataaatttaccggtggcattagccagcttgatgacgcggcagagcaaaaatgtattctggaatcaatccagcaactcaaagttaaaacgtcgtcccccgaccttgctattggacgtttgagcggcggcaatcagcaaaaagcgatcctcgctcgctgtctgttacttaacccgcgcattctcattcttgatgaacccaccaggggtatcgatattggcgcgaaatacgagatctacaaattaattaaccaactcgtccagcagggtattgccgttattgtcatctcttccgaattacctgaagtgctcggccttagcgatcgtgtactggtgatgcatgaagggaaactaaaagccaacctgataaatcataacctgactcaggagcaggtgatggaagccgcattgaggagcgaacatcatgtcgaaaagcaatccgtctga

SEQ ID NO: 8 - protein, 513 aa, Escherichia coli
MPYLLEMKNITKTFGSVKAIDNVCLRLNAGEIVSLCGENGSGKSTLMKVLCGIYPHGSYEGEIIFAGEEIQASHIRDTERKGIAIIHQELALVKELTVLENIFLGNEITHNGIMDYDLMTLRCQKLLAQVSLSISPDTRVGDLGLGQQQLVEIAKALNKQVRLLILDEPTASLTEQETSILLDIIRDLQQHGIACIYISHKLNEVKAISDTICVIRDGQHIGTRDAAGMSEDDIITMMVGRELTALYPNEPHTTGDEILRIEHLTAWHPVNRHIKRVNDVSFSLKRGEILGIAGLVGAGRTETIQCLFGVWPGQWEGKIYIDGKQVDIRNCQQAIAQGIAMVPEDRKRDGIVPVMAVGKNITLAALNKFTGGISQLDDAAEQKCILESIQQLKVKTSSPDLAIGRLSGGNQQKAILARCLLLNPRILILDEPTRGIDIGAKYEIYKLINQLVQQGIAVIVISSELPEVLGLSDRVLVMHEGKLKANLINHNLTQEQVMEAALRSEHHVEKQSV

SEQ ID NO: 9 - DNA, 1182 bp, Escherichia coli
  CDS (1)..(1182)
atgtcgaaaagcaatccgtctgaagtgaaattggccgtaccgacatccggtggcttctccgggctgaaatcactgaatttgcaggtcttcgtgatgattgcagctatcatcgcaatcatgctgttctttacctggaccaccgatggtgcctacttaagcgcccgtaacgtctccaacctgttacgccagaccgcgattaccggcatcctcgcggtaggaatggtgttcgtcataatttctgctgaaatcgacctttccgtcggctcaatgatggggctgttaggtggcgtcgcggcgatttgtgacgtctggttaggctggcctttgccacttaccatcattgtgacgctggttctgggactgcttctcggtgcctggaacggatggtgggtcgcgtaccgtaaagtcccttcatttattgtcaccctcgcgggcatgttggcatttcgcggcatactcattggcatcaccaacggcacgactgtatcccccaccagcgccgcgatgtcacaaattgggcaaagctatctccccgccagtaccggcttcatcattggcgcgcttggcttaatggcttttgttggttggcaatggcgcggaagaatgcgccgtcaggctttgggtttacagtctccggcctctaccgcagtagtcggtcgccaggctttaaccgctatcatcgtattaggcgcaatctggctgttgaatgattaccgtggcgttcccactcctgttctgctgctgacgttgctgttactcggcggaatgtttatggcaacgcggacggcatttggacgacgcatttatgccatcggcggcaatctggaagcagcacgtctctccgggattaacgttgaacgcaccaaacttgccgtgttcgcgattaacggattaatggtagccatcgccggattaatccttagttctcgacttggcgctggttcaccttctgcgggaaatatcgccgaactggacgcaattgcagcatgcgtgattggcggcaccagcctggctggcggtgtgggaagcgttgccggagcagtaatgggggcatttatcatggcttcactggataacggcatgagtatgatggatgtaccgaccttctggcagtatatcgttaaaggtgcgattctgttgctggcagtatggatggactccgcaaccaaacgccgttcttga

SEQ ID NO: 10 - protein, 393 aa, Escherichia coli
MSKSNPSEVKLAVPTSGGFSGLKSLNLQVFVMIAAIIAIMLFFTWTTDGAYLSARNVSNLLRQTAITGILAVGMVFVIISAEIDLSVGSMMGLLGGVAAICDVWLGWPLPLTIIVTLVLGLLLGAWNGWWVAYRKVPSFIVTLAGMLAFRGILIGITNGTTVSPTSAAMSQIGQSYLPASTGFIIGALGLMAFVGWQWRGRMRRQALGLQSPASTAVVGRQALTAIIVLGAIWLLNDYRGVPTPVLLLTLLLLGGMFMATRTAFGRRIYAIGGNLEAARLSGINVERTKLAVFAINGLMVAIAGLILSSRLGAGSPSAGNIAELDAIAACVIGGTSLAGGVGSVAGAVMGAFIMASLDNGMSMMDVPTFWQYIVKGAILLLAVWMDSATKRRS

SEQ ID NO: 11 - DNA, 1179 bp, Escherichia coli
  CDS (1)..(1179)
atgtttactaaacgtcaccgcatcacattactgttcaatgccaataaagcctatgaccggcaggtagtagaaggcgtaggggaatatttacaggcgtcacaatcggaatgggatattttcattgaagaagatttccgcgcccgcattgataaaatcaaggactggttaggagatggcgtcattgccgacttcgacgacaaacagatcgagcaagcgctggctgatgtcgacgtccccattgttggggttggcggctcgtatcaccttgcagaaagttacccacccgttcattacattgccaccgataactatgcgctggttgaaagcgcatttttgcatttaaaagagaaaggcgttaaccgctttgctttttatggtcttccggaatcaagcggcaaacgttgggccactgagcgcgaatatgcatttcgtcagcttgtcgccgaagaaaagtatcgcggagtggtttatcaggggttagaaaccgcgccagagaactggcaacacgcgcaaaatcggctggcagactggctacaaacgctaccaccgcaaaccgggattattgccgttactgacgcccgagcgcggcatattctgcaagtatgtgaacatctacatattcccgtaccggaaaaattatgcgtgattggcatcgataacgaagaactgacccgctatctgtcgcgtgtcgccctttcttcggtcgctcagggcgcgcggcaaatgggctatcaggcggcaaaactgttgcatcgattattagataaagaagaaatgccgctacagcgaattttggtcccaccagttcgcgtcattgaacggcgctcaacagattatcgctcgctgaccgatcccgccgttattcaggccatgcattacattcgtaatcacgcctgtaaagggattaaagtggatcaggtactggatgcggtcgggatctcgcgctccaatcttgagaagcgttttaaagaagaggtgggtgaaaccatccatgccatgattcatgccgagaagctggagaaagcgcgcagtctgctgatttcaaccaccttgtcgatcaatgagatatcgcaaatgtgcggttatccatcgctgcaatatttctactctgtttttaaaaaagcatatgacacgacgccaaaagagtatcgcgatgtaaatagcgaggtcatgttgtag

SEQ ID NO: 12 - protein, 392 aa, Escherichia coli
MFTKRHRITLLFNANKAYDRQVVEGVGEYLQASQSEWDIFIEEDFRARIDKIKDWLGDGVIADFDDKQIEQALADVDVPIVGVGGSYHLAESYPPVHYIATDNYALVESAFLHLKEKGVNRFAFYGLPESSGKRWATEREYAFRQLVAEEKYRGVVYQGLETAPENWQHAQNRLADWLQTLPPQTGIIAVTDARARHILQVCEHLHIPVPEKLCVIGIDNEELTRYLSRVALSSVAQGARQMGYQAAKLLHRLLDKEEMPLQRILVPPVRVIERRSTDYRSLTDPAVIQAMHYIRNHACKGIKVDQVLDAVGISRSNLEKRFKEEVGETIHAMIHAEKLEKARSLLISTTLSINEISQMCGYPSLQYFYSVFKKAYDTTPKEYRDVNSEVML

SEQ ID NO: 13 - DNA, 23 bp, Artificial Sequence
  Description of Artificial Sequence primer
ggcaactatgcatatcttcgcgc

SEQ ID NO: 14 - DNA, 24 bp, Artificial Sequence
  Description of Artificial Sequence primer
gcgtgaatgaattggcttaggtgg

SEQ ID NO: 15 - DNA, 21 bp, Artificial Sequence
  Description of Artificial Sequence primer
cagacagcgagcgaggatcgc

SEQ ID NO: 16 - DNA, 21 bp, Artificial Sequence
  Description of Artificial Sequence primer
tgtgcggttatccatcgctgc